(19) United States
(12) Patent Application Publication
(10) Pub. No.: US 2013/0203995 A1
(43) Pub. Date: Aug. 8, 2013

(54) Process for Making a Metabotropic Glutamate Receptor Positive Allosteric Modulator - 874

(71) Applicants: Alistair Boyd, Macclesfield (GB); Mark Richard Fielding, Macclesfield (GB); James Gair Ford, Macclesfield (GB); Lianne Frodsham, Macclesfield (GB); Michael D. Golden, Macclesfield (GB); Kevin William Leslie, Macclesfield (GB); Ben Mckeever-Abbas, Macclesfield (GB); Paula Tomlin, Macclesfield (GB)

(72) Inventors: Alistair Boyd, Macclesfield (GB); Mark Richard Fielding, Macclesfield (GB); James Gair Ford, Macclesfield (GB); Lianne Frodsham, Macclesfield (GB); Michael D. Golden, Macclesfield (GB); Kevin William Leslie, Macclesfield (GB); Ben Mckeever-Abbas, Macclesfield (GB); Paula Tomlin, Macclesfield (GB)

(73) Assignee: ASTRAZENECA AB, Sodertalje (SE)

(21) Appl. No.: 13/519,398

(22) PCT Filed: Jan. 4, 2011
(86) PCT No.: PCT/SE11/50002

§ 371 Date: Oct. 3, 2012

Related U.S. Application Data
(60) Provisional application No. 61/293,012, filed on Jan. 7, 2010.

(51) Int. Cl. C07D 413/10

(52) U.S. Cl. CPC C07D 413/10
USPC 544/367

(57) ABSTRACT

Processes for making 7-methyl-5-(3piperazin-1-ylmethyl-[1,2,4]oxadiazol-5-yl)-2-(4-trifluoromethoxybenzyl)-2,3-dihydroisoindol-1-one

### BACKGROUND

The present invention relates to methods for the preparation of an mGluR2 positive allosteric modulator.

The metabotropic glutamate receptors (mGluR) constitute a family of GTP-binding-protein (G-protein) coupled receptors that are activated by glutamate, and have important roles in synaptic activity in the central nervous system, including neural plasticity, neural development and neurodegeneration.

Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels (Schoepp et al., 1993, Trends Pharmacol. Sci., 14:13; Schoepp, 1994, Neurochem. Int., 24:439; Pin et al., 1995, Neuropharmacology 34:1; Bordi & Ugolini, 1999, Prog. Neurobiol. 59:55).

Eight mGluR subtypes have been identified, which are divided into three groups based upon primary sequence similarity, signal transduction linkages, and pharmacological profile. Group-I includes mGluR1 and mGluR5, which activate phospholipase C and the generation of an intracellular calcium signal. The Group-II (mGluR2 and mGluR3) and Group-III (mGluR4, mGluR6, mGluR7, and mGluR8) mGluRs mediate an inhibition of adenylyl cyclase activity and cyclic AMP levels. For a review, see Pin et al., 1999, Eur. J. Pharmacol., 375:277-294.

Activity of mGluR family receptors are implicated in a number of normal processes in the mammalian CNS, and are important targets for compounds for the treatment of a variety of neurological and psychiatric disorders. Activation of mGluRs is required for induction of hippocampal long-term potentiation and cerebellar long-term depression (Bashir et al., 1993, Nature, 363:347; Bortolotto et al., 1994, Nature, 368:740; Aiba et al., 1994, Cell, 79:365; Aiba et al., 1994, Cell, 79:377). A role for mGluR activation in nociception and analgesia also has been demonstrated (Meller et al., 1993, Neuroreport, 4: 879; Bordi & Ugolini, 1999, Brain Res., 871:223). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex (Nakanishi, 1994, Neuron, 13:1031; Pin et al., 1995, Neuropharmacology, supra; Knopfel et al., 1995, J. Med. Chem., 38:1417).

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### DESCRIPTION OF THE INVENTION

The schemes and processes described herein illustrate the preparation of 7-methyl-5-(3-piperazin-1-ylmethyl-[1,2,4]oxadiazol-5-yl)-2-(4-trifluoromethoxybenzyl)-2,3-dihydroisoindol-1-one, having the structure below:

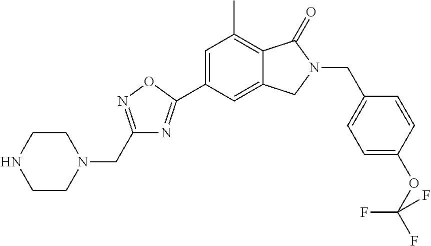 [SMILES: Cc1cc(-c2nc(CN3CCNCC3)no2)cc2c1C(=O)N(Cc1ccc(OC(F)(F)F)cc1)C2]

It will be understood by those of skill in the art that the compound made by a process described herein may exist in solvated, for example hydrated, as well as unsolvated forms. It will further be understood that the present invention encompasses all such solvated forms of the compound. Still further, it will be understood by those of skill in the art that the compound may be made by varying, or modifying, the process described herein. Particularly, those of skill in the art will understand that, as examples, solvents, temperatures or specific reagents may be changed or varied and that such changes are within the scope of the present invention.

Within the scope of the invention are also salts of the compound. Generally, pharmaceutically acceptable salts of compound made by the process of the present invention are obtained using standard procedures well known in the art.

In one embodiment of the present invention, the compound may be a pharmaceutically acceptable salt or a solvate thereof. Particularly, a pharmaceutically acceptable salt may be an acidic addition salt such as a hydrochloride, hydrobromide, phosphate, acetate, fumarate, maleate, tartrate, citrate, methanesulphonate or p-toluenesulphonate.

The invention is illustrated by way of the following schemes and examples, which describe several embodiments of the process of the invention. The synthetic schemes and the synthetic procedures provided herein are provided by way of illustration and are not to be construed as limiting the invention. It will be clear to those skilled in the art that the compound or its intermediates may be prepared by modifications of the processes described.

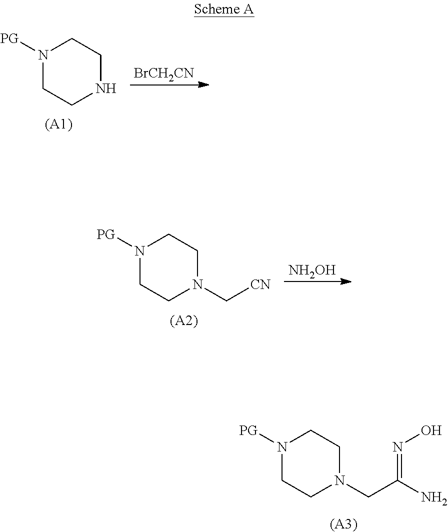

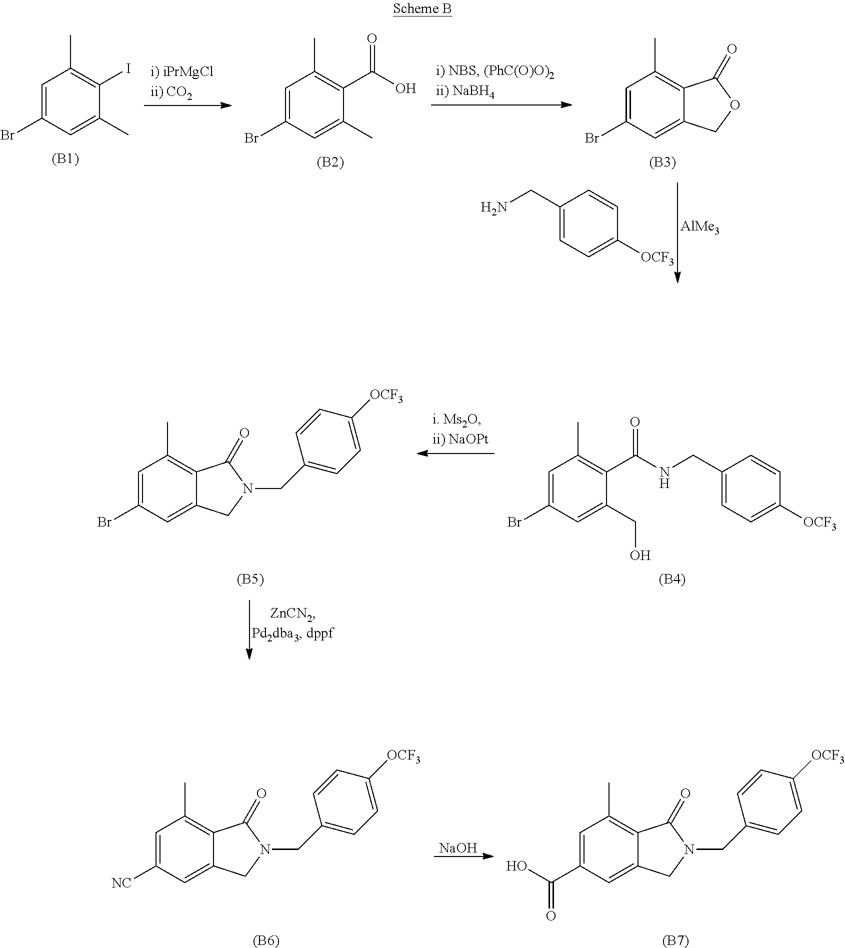

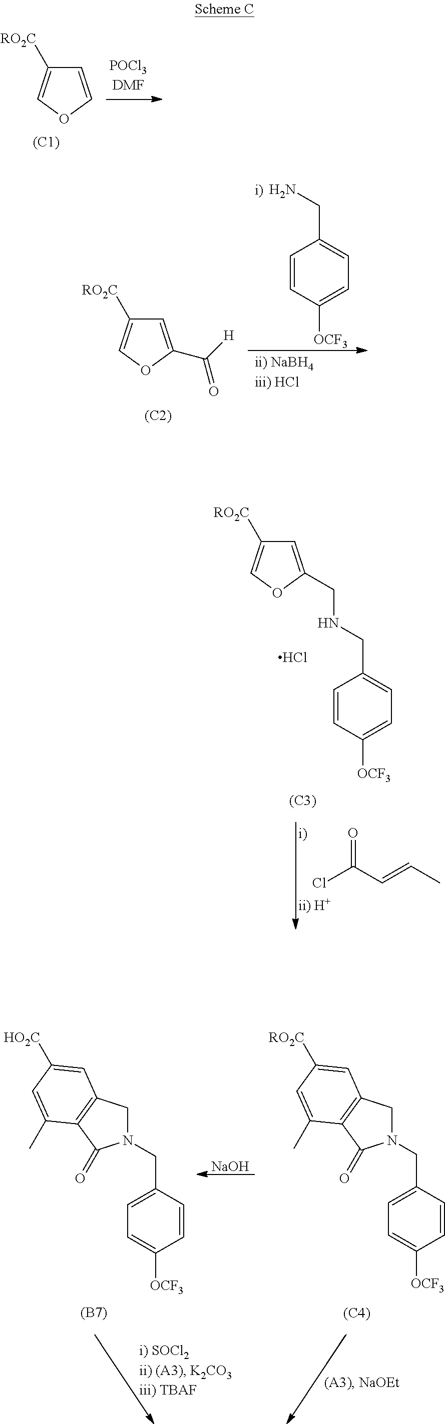

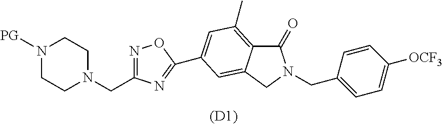

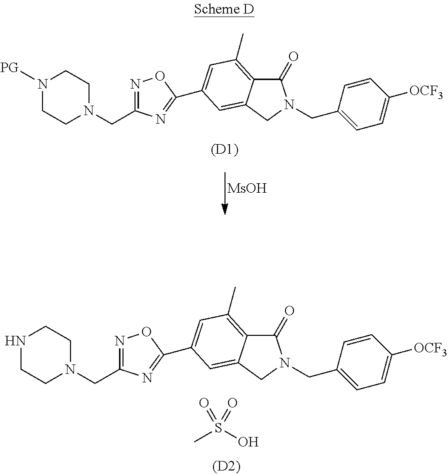

### DEFINITIONS

The following definitions are used in the schemes and descriptions herein:

NB from schemes, please check and correct where necessary
“PG”—Protecting Group, for example a BOC group

### “BrCH₂CN”—Bromoacetonitrile

### “NH₂OH”—Hydroxylamine

“iPrMgCl”—Iso-propylmagnesium chloride

### “NBS”—N-Bromosuccinimide

“(PhC(CO)O)₂”—Benzoyl peroxide

### “AlMe₃”—Trimethylaluminium

“Ms₂O”—Methanesulphonyl anhydride
“NaOPt”—Sodium t-amylate
“ZnCN₂”—Zinc cyanide
“R”—A short chain alkyl group, for example, ethyl
“POCl₃”—Phosphorous oxychloride
“DMF”—Dimethyl formamide
“NaBH₄”—Sodium borohydride

### “SOCl₂”—Thionyl Chloride

“NaOEt”—Sodium ethoxide
“TBAF”—Tetrabutylammonium fluoride
“MsOH”—Methane sulfonic acid
“rel vol” or “rel vols”—Relative volumes

For all the Examples and processes described herein amounts of materials, solvents, volumes, temperatures and times are exemplary and are not to be taken as limiting the manner in which such reactions may be performed or the scope of the invention. Variations in amounts of materials, solvents, volumes, temperatures and times may be made while carrying out the processes described and while practicing the invention described herein.

### Example 1

### Preparation of 4-(N-hydroxycarbamimidoylmethyl)-piperazine-1-carboxylic acid tert-butyl ester (A3a)

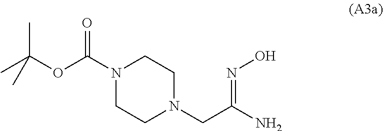 [SMILES: CC(C)(C)OC(=O)N1CCN(C/C(N)=N/O)CC1]

The title compound may be prepared according to Scheme A, as follows. Bromoacetonitrile (1.20 mol eq) may be charged to a cooled solution of tert-butyl piperazine-1-carboxylate (Ala) (1.00 mol eq) and tetramethylguanidine (1.45 mole eq) in tetrahydrofuran (3.6 rel vols). When the reaction is complete the mixture may be warmed, 2-methyltetrahydrofuran (5.0 rel vols) added and then washed with water (1.50 rel vols). The organic solution may be concentrated by vacuum distillation before methanol (1.10 rel vols) is added and the mixture cooled. Hydroxylamine hydrochloride (1.70 mol eq), tetramethylguanidine (1.70 mol eq) and water (0.25 mol eq) may be added, and the mixture heated. When the reaction is complete the mixture may be charged with sodium chloride (0.15 rel wt) and allowed to separate into 2 phases. The lower phase may be removed and back-extracted twice with 2-methyltetrahydrofuran (1.40 rel vols). The combined organic phases may be mixed with heptane (8.0 rel vols), cooled, and seeded. The mixture may be held before cooling further and charging more heptane. The title compound (A3a) may be recovered by filtration, washed once with cold water and twice with heptane and then dried under vacuum (69%).

¹H NMR (400 MHz, CDCl₃) δ: 1.46 (s, 9H), 2.42 (m, 4H), 3.00 (s, 2H), 3.43 (m, 4H), 5.10 (s, 2H).

### Example 2

### Preparation of 4-bromo-2,6-dimethylbenzoic acid (B2)

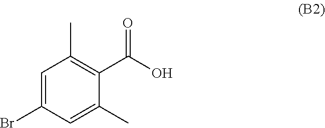

The title compound may be prepared according to Scheme B, as follows. A solution of 5-bromo-2-iodo-1,3-dimethyl-benzene (B1) (1.0 mol eq) in methyl-tert-butyl ether (6.0 rel vols) may be added to iso-propylmagnesium chloride (2.0 mol eq) in methyl-tert-butyl ether (2.0 rel vols), maintaining <0° C. Carbon dioxide gas may be added until reaction is shown to be complete. 2N aqueous hydrochloric acid may be added (4 rel vols) to quench the reaction, the phases can be separated and the aqueous phase discarded. The product may be extracted into 1M aqueous sodium hydroxide solution (6.5 rel vols), then washed with methyl tert-butyl ether (4 rel vols). The title compound (B2) may be precipitated by the addition of 2N aq hydrochloric acid (4.5 rel vols) before being filtered, washed with water then heptane and dried to yield a white crystalline solid (85%).

¹H NMR (400 MHz, CDCl₃) δ_{H} 2.28 (6H, s), 7.35 (2H, s), 13.30 (1H, s).

### Example 3

### Preparation of 5-bromo-7-methyl-3H-isobenzofuran-1-one (B3)

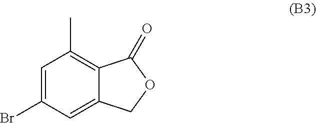

The title compound may be prepared according to Scheme B, as follows. A slurry of 4-bromo-2,6-dimethylbenzoic acid (B2) (1.0 mol eq), N-bromosuccinimide (2.5 mol eq) and benzoyl peroxide (0.1 mol eq) in chlorobenzene (10 rel vols) may be heated to ≧70° C. until the reaction is shown to be complete. A solution of 40% w/w aqueous sodium sulfite (4 rel·vol) may be added to the reaction mixture, the phases separated and the aqueous phase discarded. The organic phase may be washed with saturated aqueous sodium bicarbonate (6 rel vol). The chlorobenzene phase may be concentrated (to 3 rel vols) by vacuum distillation and dimethylacetamide added (2.5 rel vols), before adding this solution to a mixture of sodium borohydride (1.6 mol eq) in dimethylacetamide (3 rel vol). The mixture may be stirred until the reaction is shown to be complete. The mixture may be then quenched with a solution of 36% w/w hydrogen chloride (2.5 mol eq) in water (3.5 rel vol). Removing solvent by vacuum distillation will precipitate the title compound (B3) as a white crystalline solid, which may be filtered and dried (59%).

¹H NMR (400 MHz, DMSO) δ_{H} 2.57 (s, 3H), 5.32 (s, 2H), 7.60 (s, 1H), 7.73 (s, 1H).

### Example 4

### Preparation of 4-bromo-2-hydroxymethyl-6-methyl-N-(4-trifluoromethoxy-benzyl)-benzamide (B4)

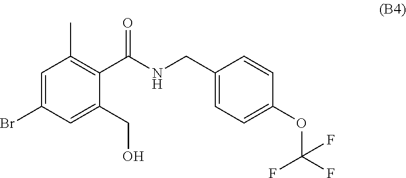 [SMILES: Cc1cc(Br)cc(CO)c1C(=O)NCc1ccc(OC(F)(F)F)cc1]

The title compound may be prepared according to Scheme B, as follows. 4-(Trifluoromethoxy)-benzylamine (1.2 mol eq) may be added to a solution of 5-bromo-7-methyl-3H-isobenzofuran-1-one (B3) (1.0 mol eq) in 2-methyltetrahydrofuran (10 rel vols) and inserted. Trimethylaluminium (1.2 mol eq) may be added and the resulting solution heated to ≧40° C. until the reaction is complete. The organic solution may be then added to a cooled solution of potassium sodium tartrate (1.5 mol eq) in water (3 rel vols) and stirred. This biphasic mixture may be separated, and the aqueous phase discarded. The organic phase may be washed with water (3.33 rel vols), then nonane (3 rel vols) may be added and the 2-methyltetrahydrofuran removed by vacuum distillation to crystallise (B4) as a white crystalline solid, which may be filtered, washed with nonane and dried (73.5%).

¹H NMR (400 MHz, DMSO) δ_{H} 2.19 (s, 3H), 4.44 (dd, 4H, 13.4 Hz, 5.78 Hz), 5.33 (t, 1H, 5.76 Hz), 7.34 (s, 1H), 7.35 (s, 2H), 7.47 (s, 1H), 7.49 (s, 2H), 8.85 (t, 1H, 5.76 Hz).

### Example 5

### Preparation of 5-bromo-7-methyl-2-(4-trifluoromethoxy-benzyl)-2,3-dihydro-isoindol-1-one (B5)

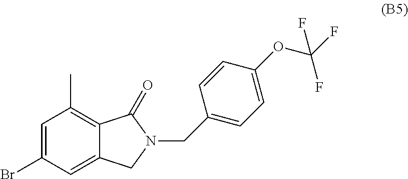 [SMILES: Cc1cc(Br)cc2c1C(=O)N(Cc1ccc(OC(F)(F)F)cc1)C2]

The title compound may be prepared according to Scheme B, as follows. Triethylamine (1.4 mol eq) may be added to 4-bromo-2-hydroxymethyl-6-methyl-N-(4-trifluoromethoxy-benzyl)-benzamide (B4) (1.0 mol eq) in 2-methyltetrahydrofuran (5 rel vols). Methanesulphonyl chloride (1.1 mol eq) may be added to this solution, maintaining <5° C. This may be stirred until the reaction is shown to be complete. Sodium tert-amylate (2.5 mol eq) may be added while maintaining the temperature at <5° C. and the reaction stirred until reaction is completed. Water (10 rel vols) may be added and the resulting biphasic mixture separated, discarding the aqueous phase. 2.7 M aqueous hydrochloric acid (4 rel vols) may be added and the phases separated, discarding the aqueous phase. The organic phase may be concentrated to dryness and the resulting wax recrystallised in heptane (4 rel vols). Filtering, washing with heptane and drying, will afford the title compound (B5) as a white solid (77%).

¹H NMR (400 MHz, DMSO) δ_{H} 2.64 (s, 3H), 4.33 (s, 2H), 4.73 (s, 2H), 7.34 (d, 2H, 8.40 Hz), 7.42 (d, 2H, 8.40 Hz), 7.47 (s, 1H), 7.59 (s, 1H).

### Example 6

### Preparation of 7-methyl-1-oxo-2-(4-trifluoromethoxy-benzyl)-2,3-dihydro-1H-isoindole-5-carbonitrile (B6)

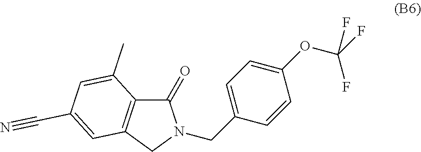 [SMILES: Cc1cc(C#N)cc2c1C(=O)N(Cc1ccc(OC(F)(F)F)cc1)C2]

The title compound may be prepared according to Scheme B, as follows. 5-Bromo-7-methyl-2-(4-trifluoromethoxy-benzyl)-2,3-dihydro-isoindol-1-one (B5) (1.0 mol eq), zinc cyanide (0.6 mol eq), 1,1′-bis(diphenylphosphino)ferrocene (0.012 mol eq), tris(dibenzylideneacetone)-dipalladium (0) (0.005 mol eq), dimethylformamide (5 rel vols) and water (0.25 mol eq) can be charged to a vessel and thoroughly inserted with nitrogen before heating to ≧90° C. until the reaction is complete. The reaction may be cooled and t-methyl butyl ether (13 rel vols) added with subsequent filtration. The organic phase obtained may be washed twice with 8.75% w/w aqueous ammonium hydroxide (14 rel vols). Heptane (14 rel vols) may be added to crystallize title compound (B6). Washing with a 9:1 heptane/MTBE mix and drying will afford the title compound (B6) as a brown solid (40%).

¹H NMR (400 MHz, DMSO) δ_{H} 2.69 (d, 3H), 4.41 (s, 2H), 4.76 (s, 2H), 7.34 (s, 1H), 7.36 (s, 1H), 7.42 (s, 1H), 7.44 (s, 1H), 7.74 (s, 1H), 7.86 (s, 1H).

### Example 7

### Preparation of 7-methyl-1-oxo-2-(4-trifluoromethoxy-benzyl)-2,3-dihydro-1H-isoindole-5-carboxylic acid (B7)

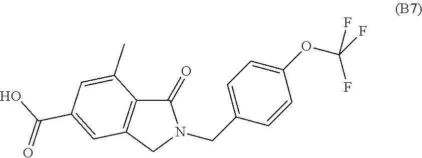 [SMILES: Cc1cc(C(=O)O)cc2c1C(=O)N(Cc1ccc(OC(F)(F)F)cc1)C2]

The title compound may be prepared according to Scheme B, as follows. 7-Methyl-1-oxo-2-(4-trifluoromethoxy-benzyl)-2,3-dihydro-1H-isoindole-5-carbonitrile (B6) may be mixed with methanol (10 rel vols) and 49% aqueous potassium hydroxide (3.0 mol eq) and heated until reaction is complete. The mixture may be concentrated under vacuum to remove the methanol and then diluted with aq. hydrochloric acid to precipitate the product, which may be extracted with iso-propyl acetate. The organic phase may be concentrated to remove solvent, then triturated with heptane, filtered and dried to afford the title compound (B7) (85%).

¹H NMR (400 MHz, DMSO) δ: 2.71 (s, 3H), 4.42 (s, 2H), 4.77 (s, 2H), 7.35 (d, 2H, 8.73 Hz), 7.43 (d, 2H, 8.73 Hz), 7.81 (s, 1H), 7.91 (s, 1H), 13.19 (s, 1H, br).

### Example 8a

### Preparation of 5-formyl-furan-3-carboxylic acid ethyl ester (C2a)

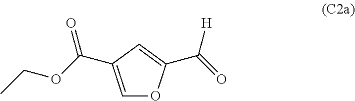

The title compound may be prepared according to Scheme C, as follows. To a solution of ethyl-3-furoate (C1a) (1 mol eq) in N,N-dimethyl formamide (4 mol eq) may be added phosphoryl chloride (4 mol eq). The resulting solution may be heated to 60° C. until reaction is deemed complete. The reaction mixture may be then added to a solution of sodium carbonate (8.25 mol eq) in water (18 rel vols). Toluene (7 rel vols) may be added, and the phases separated. The organic phase may be concentrated (to 4.2 rel vols total volume) under atmospheric pressure. This will afford a 22.1% w/w solution of the title compound (C2a) as 3.5% w/w solution in toluene (73%).

¹H NMR (400 MHz, CDCl₃) δ_{H} 1.37 (3H, t, 7.2 Hz), 4.33 (2H, q, 7.2 Hz), 7.54 (1H, m), 8.22 (1H, m) and 9.69 (1H, m).

### Example 8b

### Preparation of 5-formyl-furan-3-carboxylic acid ethyl ester (C2a)

The title compound may be prepared according to Scheme C by use of a flow reactor (for example, a 2-train Continuously Stirred Tank Reactor (CSTR)). A CSTR may be fed with ethyl-3-furoate (C1a) (1 mol eq), N,N-dimethyl formamide (3.833 mol eq) and phosphoryl chloride (4.167 mol eq). The reaction temperature may be maintained at 105° C., and the feed rates adjusted to yield a mean residence time of 2 hours. The solution leaving the reactor may be quenched into a solution of potassium bicarbonate (8.33 mol eq) in water (3.13 L/g potassium carbonate). Toluene (0.26 L/L water) may be added, and the phases separated. The organic phase may be concentrated (by 3.2 volumes) via distillation under atmospheric pressure. This will afford a 22.4% w/w solution of the title compound (C2a) in toluene.

¹H NMR (400 MHz, CDCl₃) δ_{H} 1.37 (3H, t, 7.2 Hz), 4.33 (2H, q, 7.2 Hz), 7.54 (1H, m), 8.22 (1H, m) and 9.69 (1H, m).

### Example 9

### Preparation of 5-[(4-trifluoromethoxy-benzylamino)-methyl]-furan-3-carboxylic acid ethyl ester hydrochloride (C3a)

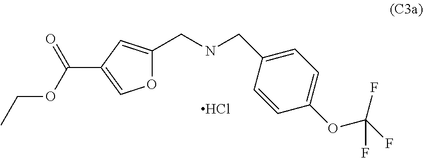 [SMILES: CCOC(=O)c1coc(CNCc2ccc(OC(F)(F)F)cc2)c1 ; Cl]

The title compound may be prepared according to Scheme C, as follows. To a 22.1% w/w solution of 5-formyl-furan-3-carboxylic acid ethyl ester (C2a) (1 mol eq) in toluene, 4-(trifluoromethoxy)-benzylamine (1.1 mol eq) may be added, and the reaction stirred until the reaction is complete. Sodium borohydride (1.25 mol eq) may be added and the resulting slurry cooled to ≧−5° C. Ethanol (16.0 rel·vol.) may be added and the resulting solution quenched with aqueous hydrochloric acid (3.0 mol eq) to crystallise the title compound (C3a). This slurry may be heated to ≧60° C. and then cooled to improve the crystal form. Filtering and washing with toluene and then aqueous ethanol will afford the title compound (C3a) as a crystalline white solid (85%).

¹H NMR (400 MHz, DMSO) δ_{H} 1.28 (3H, t), 4.24 (6H, m), 6.97 (1H, d), 7.44 (1H, d), 7.70 (1H, d), 8.46 (1H, d).

### Example 10

### Preparation of 7-methyl-1-oxo-2-(4-trifluoromethoxy-benzyl)-2,3-dihydro-1H-isoindole-5-carboxylic acid ethyl ester (C4a)

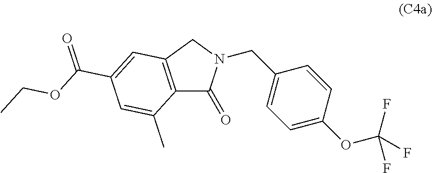 [SMILES: CCOC(=O)c1cc(C)c2c(c1)CN(Cc1ccc(OC(F)(F)F)cc1)C2=O]

The title compound may be prepared according to Scheme C, as follows. Crotonoyl chloride (1.05 mol eq) may be added to a mixture of 5-[(4-trifluoromethoxy-benzylamino)-methyl]-furan-3-carboxylic acid ethyl ester hydrochloride (C3a) (1.0 mol eq), di-isopropylethylamine (2.5 mol eq) and toluene (10 rel vols) at ambient temperature. When the reaction is complete, the mixture may be washed with water (5 rel vols), concentrated (to 5 rel vols total volume) and heated to reflux for >12 hours. Methanesulphonic acid (1.0 mol eq) may be added, and the reaction then heated to reflux under Dean & Stark conditions until reaction is deemed complete. The solution may be then washed with aqueous sodium hydroxide solution (5 rel vols) then water (5 rel vols). Heptane (10 rel vols) may be added, and the mixture cooled to permit crystallization to occur. The mixture may be filtered and the recovered solid dried to afford the title compound (C4a) as a white crystalline solid (73%).

¹H NMR (400 MHz, CDCl₃) δ: 1.40 (t, 3H, 7.12 Hz), 2.81 (s, 3H), 4.27 (s, 2H), 4.39 (q, 2H, 7.12 Hz), 4.78 (s, 2H), 7.18 (d, 2H, 8.60 Hz), 7.35 (d, 2H, 8.19 Hz), 7.87 (s, 1H), 7.90 (s, 1H).

### Example 11

### Preparation of 7-methyl-1-oxo-2-[4-(trifluoromethoxy)-benzyl]-2,3-dihydro-1H-isoindole-5-carboxylic acid (B7)

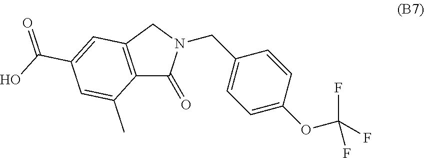 [SMILES: Cc1cc(C(=O)O)cc2c1C(=O)N(Cc1ccc(OC(F)(F)F)cc1)C2]

The title compound may be prepared as according to Scheme C, as follows. 7-Methyl-1-oxo-2-(4-trifluoromethoxy-benzyl)-2,3-dihydro-1H-isoindole-5-carboxylic acid ethyl ester (C4a) may be mixed with 2-propanol (4 rel vols) and 49% aqueous potassium hydroxide (1.5 mol eq) and heated until reaction is complete. 36% w/w aqueous hydrochloric acid (2.00 mol eq), may be added, the reaction mixture cooled to ambient temperature and water (5 rel vols) added. The title compound (B7) may be recovered by filtration and dried (98%).

¹H NMR (400 MHz, DMSO) δ: 2.71 (s, 3H), 4.42 (s, 2H), 4.77 (s, 2H), 7.35 (d, 2H, 8.73 Hz), 7.43 (d, 2H, 8.73 Hz), 7.81 (s, 1H), 7.91 (s, 1H), 13.19 (s, 1H, br).

### Example 12a

### Preparation of 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester (D1a)

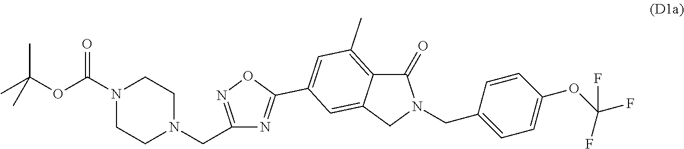 [SMILES: Cc1cc(-c2nc(CN3CCN(C(=O)OC(C)(C)C)CC3)no2)cc2c1C(=O)N(Cc1ccc(OC(F)(F)F)cc1)C2]

The title compound may be prepared according to Scheme C, as follows. 4-(N-Hydroxycarbamimidoylmethyl)-piperazine-1-carboxylic acid tert-butyl ester (A3a) (1.0 mol eq) and 7-methyl-1-oxo-2-(4-trifluoromethoxy-benzyl)-2,3-dihydro-1H-isoindole-5-carboxylic acid ethyl ester (C4a) (1.0 mol eq) can be mixed in acetonitrile (14 rel vols) at 60° C. Sodium ethoxide (0.2 mols) may be added over 20 mins and the reaction held at 60° C. until complete. Water (10 rel vols) may be added and the mixture cooled to ambient temperature. The title compound (D1a) may be recovered by filtration and washed with water before being dried (84%).

¹H NMR (400 MHz, CDCl₃) δ: 1.46 (9H, s), 2.58 (4H, m), 2.84 (3H, s), 3.50 (4H, m), 3.79 (2H, s), 4.33 (2H, s), 4.81 (2H, s), 7.20 (2H, m), 7.36 (2H, m), 7.99 (1H, s), 8.04 (1H, s).

### Example 12b

### Preparation of 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester (D1a)

The title compound may be prepared according to Scheme C, as follows. A slurry of 7-methyl-1-oxo-2-[4-(trifluoromethoxy)-benzyl]-2,3-dihydro-1H-isoindole-5-carboxylic acid (B7) (1.00 mol eq) in toluene (8.0 rel vols) may be heated and thionyl chloride (1.50 mol eq) added. When the reaction is complete, excess thionyl chloride and toluene can be removed by atmospheric distillation. This solution may be added to a slurry of 4-(N-hydroxycarbamimidoylmethyl)-piperazine-1-carboxylic acid tert-butyl ester (A3a) (1.1 mol eq) and potassium carbonate (1.25 mol eq) in 2-methyl tetrahydrofuran (19.0 rel vols). Tetrabutylammonium fluoride (1.0 mol eq) in tetrahydrofuran (0.5 rel vols) may be charged and the contents heated until the cyclization is complete. After cooling the organic phase may be washed with water before being concentrated by distillation. Methyl-t-butyl ether may be added to induce crystallization. After cooling, the title compound (D1a) may be recovered by filtration, washed with methyl-t-butyl ether and dried to constant weight in a vacuum oven.

¹H NMR (400 MHz, CDCl₃) δ: 1.46 (9H, s), 2.58 (4H, m), 2.84 (3H, s), 3.50 (4H, m), 3.79 (2H, s), 4.33 (2H, s), 4.81 (2H, s), 7.20 (2H, m), 7.36 (2H, m), 7.99 (1H, s), 8.04 (1H, s).

### Example 13

### Preparation of 7-methyl-5-(3-piperazin-1-ylmethyl-[1,2,4]oxadiazol-5-yl)-2-(4-trifluoromethoxy-benzyl)-2,3-dihydro-isoindol-1-one as a methanesulphonate (D2)

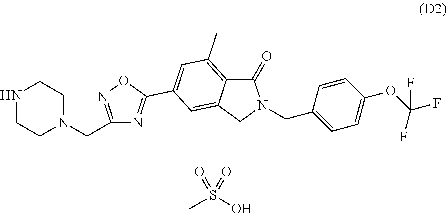

The title compound may be prepared according to Scheme D, as follows. To a solution of 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester (D1a) (0.170 mol) in a mixture of 1-butanol (250 mL) and water (30 mL) at 85° C. may be added methanesulfonic acid (0.187 mol). After addition of further water (10 mL), the reaction mixture may be held at 85° C. until reaction is complete. The reaction mixture may be cooled to 65-70° C. before conducting a screening filtration into a crystallizer. The reaction vessel and the line can be rinsed into the crystallizer with hot (80° C.) 1-butanol (1×200 mL). The resulting reaction mixture may be then held at 85° C. before addition of 1-butanol (800 mL), cooling to 78° C. and seeding with (D2) (0.1 g, 1% w/w). The reaction mixture can then be cooled to 15° C. and temperature-cycled to 65-70° C. twice, before filtration to afford the title compound (D2) which may be washed with 1-butanol (2×200 mL) before drying under vacuum to afford the title compound as a white solid (87.38 g, 88.0% yield).

¹H NMR (400 MHz, DMSO) δ: 2.36 (3H, s), 2.81 (4H, m), 3.15 (4H, m), 3.91 (2H, s), 4.46 (2H, s), 4.78 (2H, s), 7.37 (2H, m), 7.45 (2H, m), 8.00 (1H, m), 8.12 (1H, m) and 8.59 (1H, br s).

1. A process for preparing 7-methyl-5-(3-piperazin-1-ylmethyl-[1,2,4]oxadiazol-5-yl)-2-(4-trifluoromethoxybenzyl)-2,3-dihydroisoindol-1-one, having the structure below: 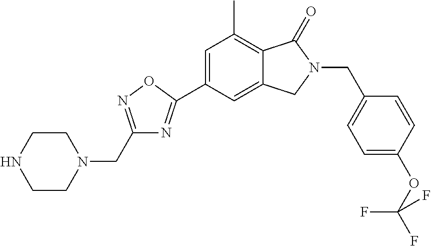 [SMILES: Cc1cc(-c2nc(CN3CCNCC3)no2)cc2c1C(=O)N(Cc1ccc(OC(F)(F)F)cc1)C2]
or an acid salt thereof, the process comprising:
reacting tert-butyl piperazine-1-carboxylate with bromoacetonitrile, in an organic solvent in the presence of a base and subsequent reaction with hydroxylamine or an acid salt thereof to form 4-(N-hydroxycarbamimidoylmethyl)-piperazine-1-carboxylic acid tert-butyl ester;
reacting 5-bromo-2-iodo-1,3-dimethyl-benzene in an ether solvent with a Grignard reagent and carbon dioxide gas to form 4-bromo-2,6-dimethylbenzoic acid;
heating 4-bromo-2,6-dimethylbenzoic acid with N-bromosuccinimide and a radical initiator in an organic solvent and subsequent reduction of over-brominated products with a reducing agent to form 5-bromo-7-methyl-3H-isobenzofuran-1-one;
heating 5-bromo-7-methyl-3H-isobenzofuran-1-one with 4-(trifluoromethoxy)-benzylamine in an anhydrous solvent with trimethylaluminium to form 4-bromo-2-hydroxymethyl-6-methyl-N-(4-trifluoromethoxy-benzyl)-benzamide;
reacting 4-bromo-2-hydroxymethyl-6-methyl-N-(4-trifluoromethoxy-benzyl)-benzamide with methanesulphonyl chloride and a base in an organic solvent to form 5-bromo-7-methyl-2-(4-trifluoromethoxy-benzyl)-2,3-dihydro-isoindol-1-one;
heating 5-bromo-7-methyl-2-(4-trifluoromethoxy-benzyl)-2,3-dihydro-isoindol-1-one in the presence of zinc cyanide, 1,1′-bis(diphenylphospino)ferrocene and tris(dibenzylideneacetone)-dipalladium in an organic solvent to form 7-methyl-1-oxo-2-(4-trifluoromethoxy-benzyl)-2,3-dihydro-1H-isoindole-5-carbonitrile;
hydrolysis of 7-methyl-1-oxo-2-(4-trifluoromethoxy-benzyl)-2,3-dihydro-1H-isoindole-5-carbonitrile in a solvent with a hydroxide solution to form 7-methyl-1-oxo-2-(4-trifluoromethoxy-benzyl)-2,3-dihydro-1H-isoindole-5-carboxylic acid;
reacting 7-methyl-1-oxo-2-(4-trifluoromethoxy-benzyl)-2,3-dihydro-1H-isoindole-5-carboxylic acid in an organic solvent with thionyl chloride or another chlorinating agent and reacting the product with tert-butyl 4-[(2Z)-2-amino-2-hydroxyimino)ethyl]-piperazine-1-carboxylate in the presence of a base to form 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester, and
decarboxylating 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester by heating with acid in a solvent, to form 7-methyl-5-(3-piperazin-1-ylmethyl-[1,2,4]oxadiazol-5-yl)-2-(4-trifluoromethoxy-benzyl)-2,3-dihydro-isoindol-1-one or an acid salt thereof.

2. A process for preparing 7-methyl-5-(3-piperazin-1-ylmethyl-[1,2,4]oxadiazol-5-yl)-2-(4-trifluoromethoxybenzyl)-2,3-dihydroisoindol-1-one, having the structure below: 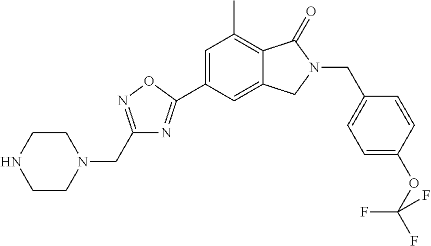 [SMILES: Cc1cc(-c2nc(CN3CCNCC3)no2)cc2c1C(=O)N(Cc1ccc(OC(F)(F)F)cc1)C2]
or an acid salt thereof, the process comprising:
reacting tert-butyl piperazine-1-carboxylate with bromoacetonitrile, in an organic solvent in the presence of a base and subsequent reaction with hydroxylamine or an acid salt thereof to form 4-(N-hydroxycarbamimidoylmethyl)-piperazine-1-carboxylic acid tert-butyl ester;
formylating ethyl-3-furoate with a Vilsmeier reagent formed by reaction of phosphoryl chloride with dimethylformamide or another formylating agent to form 5-formyl-furan-3-carboxylic acid ethyl ester;
reacting 5-formyl-furan-3-carboxylic acid ethyl ester in an organic solvent with 4-(trifluoromethoxy)-benzylamine and reducing the product to form 5-[(4-trifluoromethoxy-benzylamino)-methyl]-furan-3-carboxylic acid ethyl ester or an acid salt thereof;
reacting 5-[(4-trifluoromethoxy-benzylamino)-methyl]-furan-3-carboxylic acid ethyl ester or an acid salt thereof with crotonoyl chloride in the presence of a base, with subsequent heating or other conditions so as to affect a Diels Alder transformation and subsequent dehydration of the product with an acid or base to form 7-methyl-1-oxo-2-(4-trifluoromethoxy-benzyl)-2,3-dihydro-1H-isoindole-5-carboxylic acid ethyl ester;
reacting 7-methyl-1-oxo-2-(4-trifluoromethoxy-benzyl)-2,3-dihydro-1H-isoindole-5-carboxylic acid ethyl ester with tert-butyl 4-[(2Z)-2-amino-2-hydroxyimino)ethyl]-piperazine-1-carboxylate in an organic solvent with a base to form 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester, and
decarboxylating 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester by heating with acid in an organic solvent, to form 7-methyl-5-(3-piperazin-1-ylmethyl-[1,2,4]oxadiazol-5-yl)-2-(4-trifluoromethoxy-benzyl)-2,3-dihydro-isoindol-1-one or an acid salt thereof.

3. A compound selected from the group consisting of: 5-bromo-7-methyl-3H-isobenzofuran-1-one; 4-bromo-2-hydroxymethyl-6-methyl-N-(4-trifluoromethoxy-benzyl)-benzamide; 5-[(4-trifluoromethoxy-benzylamino)-methyl]-furan-3-carboxylic acid or an alkyl ester thereof, and 7-methyl-1-oxo-2-(4-trifluoromethoxy-benzyl)-2,3-dihydro-1H-isoindole-5-carboxylic acid or an alkyl ester thereof.

4-7. (canceled)
